(19) United States
(12) Patent Application Publication
(10) Pub. No.: US 2014/0257108 A1
(43) Pub. Date: Sep. 11, 2014

(54) PRECONFIGURED ULTRASOUND CONDUCTIVE MEDIUM

(71) Applicant: G. Chad MORGAN, Chattanooga, TN (US)

(72) Inventor: G. Chad MORGAN, Chattanooga, TN (US)

(21) Appl. No.: 14/181,130

(22) Filed: Feb. 14, 2014

Related U.S. Application Data
(60) Provisional application No. 61/765,392, filed on Feb. 15, 2013.
(60) Provisional application No. 61/765,376, filed on Feb. 15, 2013.
(60) Provisional application No. 61/919,233, filed on Dec. 20, 2013.

(51) Int. Cl. A61B 8/00

(52) U.S. Cl. CPC A61B 8/4281; A61B 8/4455
USPC 600/459

(57) ABSTRACT

Embodiments associated with ultrasound conductive medium are described. In one embodiment, an ultrasound conductive medium is formed into a preconfigured shape that is a structure configured to attach over a head of an ultrasound device.

### CROSS REFERENCE TO RELATED APPLICATIONS

This patent disclosure claims the benefit of U.S. Provisional Patent application Ser. No. 61/765,392 entitled “Preconfigured gel with infused drug for ultrasound” filed on Feb. 15, 2013, and U.S. Provisional Patent Application Ser. No. 61/765,376 entitled “Preconfigured gel for ultrasound” filed on Feb. 15, 2013, and U.S. Provisional Patent Application Ser. No. 61/919,233 entitled “Gel with locking element and gel adaptor” filed on Dec. 20, 2013, which are all hereby wholly incorporated by reference in their entirety.

### BACKGROUND

Ultrasound devices operate with frequencies from 0 to 200 mHz up to several gigahertz and are used in many different fields. In the medical field, ultrasound can be used for therapeutic procedures and imaging of internal structures. For example, ultrasound can be applied to a patient's skin to stimulate the tissue beneath the skin's surface using very high frequency sound waves.

Ultrasound is applied using a device that includes a transducer or applicator that is put in contact with a patient's skin. A random amount of liquid gel is dispensed on the patient's skin to cover the area and on all surfaces of the device's head. The gel functions to couple the device with the skin, to reduce friction, and to assist transmission of the ultrasonic waves. The gel is squeezed out of a bottle and spread over the patient's skin. Since the gel is a fluid, it is difficult to contain within a desired area of the skin and the thickness of the gel cannot be controlled. Lack of a consistent and desired thickness of the gel can lead to a less than optimal ultrasound application. Gel that is too thin or too thick can affect the quality of images produced by the device, therapeutic values, and/or efficacy. Furthermore, when the ultrasound procedure is completed, the patient is required to clean up and wipe off the gel from the patient's skin. Typically, the gel is not completely removed and the cleaning process is uncomfortable.

### SUMMARY

In one embodiment of the disclosure, an ultrasound conductive medium is disclosed that is formed into a preconfigured shape that is a cap-like structure configured to attach over a head of an ultrasound device. In another embodiment, the ultrasound conductive medium is in a solidified state that maintains the preconfigured shape. In another embodiment, the ultrasound conductive medium is formed from a gel.

In another embodiment, the cap-like structure includes flexible side walls that define an internal cavity. In another embodiment, the flexible side walls include one or more protrusions formed within the internal cavity, wherein the one or more protrusions are configured to assist in securing the ultrasound conductive medium to the head of the ultrasound device once attached thereto. In another embodiment, the one or more internal protrusions are oriented and configured to mate with or abut against corresponding notches or edges in the head of the ultrasound device. In another embodiment, the cap-like structure includes sidewalls and an interface surface, wherein the interface surface has a predetermined thickness of the ultrasound conductive medium to provide a consistent amount of ultrasound conductive medium between the ultrasound device and a patient.

In another embodiment of the disclosure, an ultrasound conductive medium is disclosed that is molded into a solid form having a shape defined to cover and enclose a head of an ultrasound device. In another embodiment, the ultrasound conductive medium includes one or more sidewalls that define an internal cavity for receiving the head of the ultrasound device. In another embodiment, the one or more sidewalls include one or more protrusions, wherein the one or more protrusions are configured to assist in securing the ultrasound conductive medium to the head of the ultrasound device once attached thereto.

In another aspect, the solid form of the ultrasound conductive medium includes a predetermined thickness of ultrasound conductive medium to provide a consistent and controlled amount of the ultrasound conductive medium between a transducer of the ultrasound device and a patient. In another embodiment, the shape is a cap-like structure that includes flexible sidewalls for attaching to the head of the ultrasound device. In another embodiment, the shape is a tube shape that includes elongated sidewalls that define an internal cavity, wherein the internal cavity is configured to receive a long and narrow ultrasound probe.

In another embodiment, the cap-like structure ultrasound conductive medium includes sidewalls and an interface surface, wherein the interface surface has a predetermined thickness of the ultrasound conductive medium to provide a consistent amount of ultrasound conductive medium between the ultrasound device and a patient. In another embodiment, the ultrasound conductive medium comprises an ultrasound gel.

In another embodiment, the flexible side walls include one or more protrusions formed within the internal cavity, wherein the one or more protrusions are configured to assist in securing the ultrasound conductive medium to the head of the ultrasound device once attached thereto.

In another embodiment of the disclosure, an ultrasound device is disclosed that includes at least a handle connected to a head, wherein the head contains an ultrasound transducer; and an ultrasound conductive medium having a solidified shape, wherein the solidified shape is attach to and over the head of the ultrasound device. The ultrasound conductive medium is configured to provide an interface surface and coupling medium between the ultrasound transducer and an ultrasound target.

In another embodiment, the solidified shape is a cap-like structure comprising one or more flexible sidewalls that define an internal cavity for receiving the head of the ultrasound device. The ultrasound conductive medium is attached to move with the head when the ultrasound device is moved. In another embodiment, the one or more sidewalls includes one or more protrusions.

In another embodiment, the solidified shape comprises solidified gel and the ultrasound conductive medium is removable from the head.

### BRIEF DESCRIPTION OF THE DRAWINGS

The accompanying drawings, which are incorporated in and constitute a part of the specification, illustrate various systems, methods, and other embodiments of the disclosure. It will be appreciated that the illustrated element boundaries (e.g., boxes, or other shapes) in the figures represent one embodiment of the boundaries. In some embodiments one element may be designed as multiple elements or that multiple elements may be designed as one element. In some embodiments, an element shown as an internal component of another element may be implemented as an external component and vice versa. Furthermore, elements may not be drawn to scale.

FIGS. 1A and 1B illustrate one embodiment of a preconfigured gel from a top view and side view, respectively.

FIG. 1C illustrates another embodiment of the preconfigured gel from a side view.

FIG. 1D illustrates a 3-dimensional image of the preconfigured gel from FIG. 1B.

FIG. 2A illustrates one embodiment of an ultrasound device shown with components unassembled.

FIG. 2B illustrates the ultrasound device of FIG. 2A in an assembled view.

FIG. 3A illustrates one embodiment of an ultrasound conductive medium configured in a cap-like structure.

FIG. 3B illustrates the ultrasound conductive medium in FIG. 3A in cross-section.

FIG. 3C illustrates another embodiment of the ultrasound conductive medium of FIGS. 3A and 3B including an internal protrusion.

FIGS. 4A and 4B illustrates the ultrasound conductive medium of FIG. 3A being attached to a head of an ultrasound device to cover the head.

FIG. 5 illustrates another embodiment of an ultrasound conductive medium that is preconfigured with extended sidewalls.

FIG. 6 illustrates another embodiment of an ultrasound conductive medium that is preconfigured as a rectangular cap.

FIG. 7 illustrates a top surface view of another embodiment of an ultrasound conductive medium that is preconfigured as a rectangular cap with rounded corners.

FIG. 8 illustrates a top surface view of another embodiment of an ultrasound conductive medium that is preconfigured with a generally oval shape.

### DETAILED DESCRIPTION

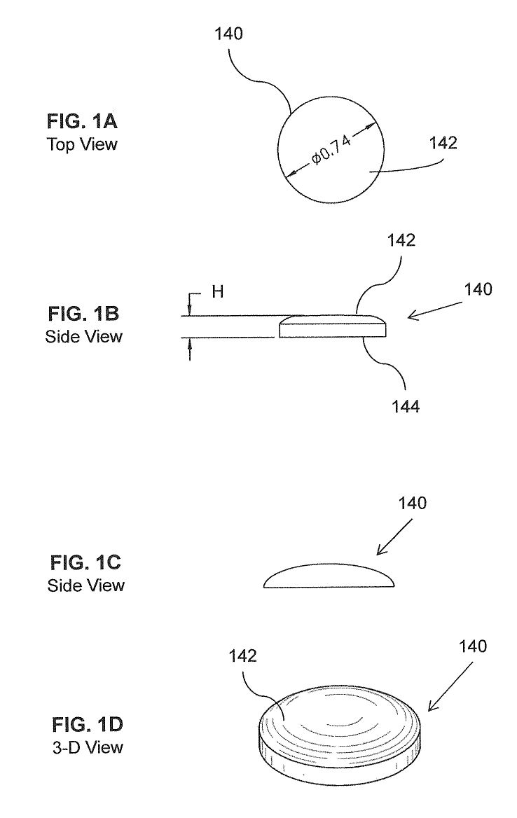

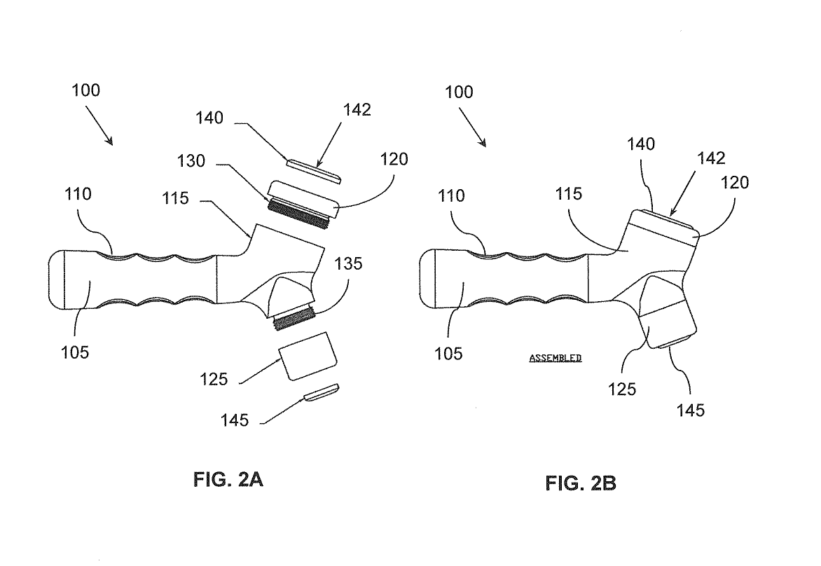

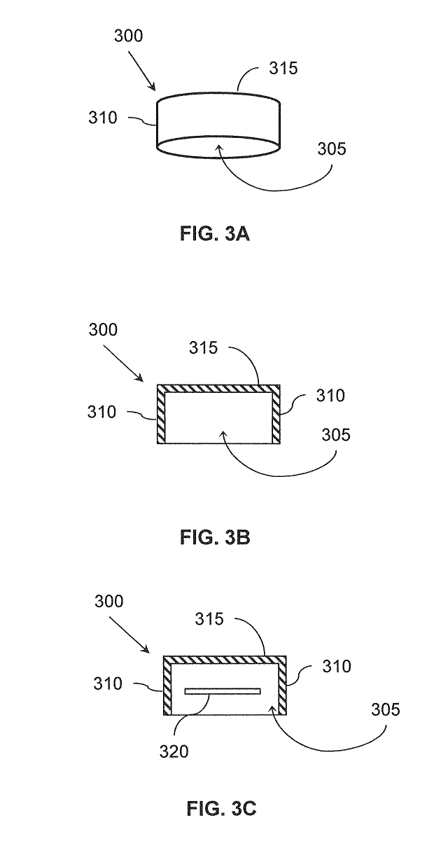

Embodiments of preconfigured gel are disclosed herein that are used for ultrasound devices. In one embodiment, ultrasound gel is preconfigured into a predefined shape (herein also referred to as a “gel shot”, which is an individual piece of gel). In general, gel refers to an ultrasound conductive medium. As will be described with reference to FIGS. 1-2, the predefined shape is made to correspond to a recessed cavity of an ultrasound device in which the preconfigured gel is inserted. In one embodiment, the preconfigured gel is infused with a drug to be delivered during an ultrasound procedure (e.g., transdermal delivery). In one embodiment, the drug is Lidocaine or other anesthetic. In another embodiment, as will be described with reference to FIGS. 3-8, gel is preconfigured into a defined shape to cover the head of an ultrasound device (herein also referred to as a “gel cover” or “gel cap”).

In one embodiment, the preconfigured gel (gel shot or gel cover) is formed from an aqueous material that functions as a conductive medium and coupling medium for ultrasound energy. The aqueous material is for example a liquid or jelly-like substance/composition that is molded into a desired shape and processed to retain its shape as a solid form (e.g., via curing, heating, cooling, or other process). For example, the gel composition is processed to solidify the gel to a desired extent so that its shape is set. In different embodiments, the preconfigured gel can be solidified to different degrees or ranges as desired such as being a soft and flexible object, being a rigid object, or any state in between (e.g., semi-rigid and/or flexible structure).

In one embodiment, the gel shot or gel cover is formed to retain its shape and have memory. Once the shape is set, which has predetermined amount of gel, the preconfigured gel provides a consistent amount and thickness of gel for an ultrasound procedure. The gel shot or gel cover is formed in advance so that it is ready for use with an ultrasound device and provides a premeasured and consistent amount of gel. After being used in an ultrasound procedure, the preconfigured gel is removed from the ultrasound device and replaced with a new piece of preconfigured gel. Of course, the same piece of gel may be used for multiple procedures since it is attached to the ultrasound device and can be moved. However, there may be issues with sterility from multiple uses that may not be desired.

With reference to FIGS. 1A and 1B, one embodiment of preconfigured gel 140 is illustrated, which is in a solidified state. FIG. 1A shows a top view of the gel 140 and FIG. 1B shows a side view. FIG. 1C illustrates a side view of another embodiment of the gel 140 with a different shape. FIG. 1D illustrates a 3-dimensional image of the preconfigured gel 140 where the gel 140 represents the shape of FIGS. 1A and 1B. Of course, other shapes can be formed.

In one embodiment, the preconfigured gel 140 is formed with a circular top surface 142 and bottom surface 144 and has a general configuration of a disk/pad. This shape would correspond to a head of ultrasound device that is generally round. The preconfigured gel 140 includes a thickness or height H. In one embodiment, the height H of the gel 140 is about 0.15 inches and the diameter is about 0.74 inches. In another embodiment, the diameter may be about 1.17 inches for use with ultrasound devices that have a larger circular head. Of course, other dimensions may be implemented.

The shape and size of the preconfigured gel 140 may be defined to correspond to a gel receiving cavity of an ultrasound device in which the gel 140 is to function with. Of course, the gel 140 may be implemented in other shapes and sizes (e.g., 3-dimensional oval or polygon, other shapes with curved sides, flat sides, rectangular shape, and so on to correspond to the shape of an ultrasound head). The top surface 142 may be flat (see FIG. 1B) or curved (see FIG. 1C) in order to provide good contact with an ultrasound target (e.g., a patient's skin). In other embodiments, the preconfigured gel 140 may be formed with irregular shapes such as having the top surface 142 that is larger than the bottom surface 144 (e.g., mushroom shape). In other embodiments, the gel cover may be formed in various shapes and sizes to conform to an applicable head of an ultrasound device (see FIGS. 3-8).

In one embodiment, the preconfigured gel is formed or molded into a desired shape. This may involve using molds. Multiple molds can be used to create multiple pieces of the preconfigured gel at a time. For example, a tray of patterned shapes can be used where the gel composition in a liquid form is inserted into each shape. The gel composition is then processed to solidify the gel to a desired extent so that its shape is set (e.g., the composition holds its shape, does not flow, or does not take the shape of its container when placed in the container). Of course, other processes may be used to create the preconfigured gel or cover such as injection molding, 3-D printing, and so on. In one embodiment, the molding process also applies to the gel cover embodiments of FIGS. 3-8.

### Ultrasound Device Embodiment

With reference to FIGS. 2A and 2B, in one embodiment, an ultrasound device 100 described herein includes a diaphragm configured with a recessed cavity for receiving and containing the preconfigured gel 140. In another embodiment, the diaphragm is configured as a replaceable head for an ultrasound device where the diaphragm includes an ultrasound transducer (e.g., piezoelectric crystal) and a cavity for containing the preconfigured gel 140.

FIG. 2A illustrates the ultrasound device 100 in a partially unassembled state and FIG. 2B shows the device 100 in an assembled state. The device 100 is configured as a hand-held device including an elongated handle 105 that may include one or more finger grips 110 (e.g., indentations, ridges, and so on). The handle 105 is connected to a head 115 that includes one or more sides that connect to a diaphragm. In the illustrated embodiment, the head 115 includes two sides where the first side includes diaphragm 120 that includes a transducer and the second side includes diaphragm 125 and a transducer (e.g., the device 100 includes two ultrasound heads). Of course, other types of ultrasound devices or probes may be used with the preconfigured gel described herein. Thus the embodiment of FIGS. 2A-2B are not intended to be limiting.

In one embodiment, diaphragm 120 is configured with a connector 130 that is threaded to insert and connect with a corresponding threaded socket in the head 115. Similarly, the second side of the head 115 may include a connector 135 that is threaded for connecting with a threaded socket within the diaphragm 125. In another embodiment, the connectors 130 and 135 may be configured as a quick-connect/disconnect device so that the diaphragms 120 and 125 can be connected by pushing and snapping into place or disconnected by pulling off with a small amount of force. Thus, in one embodiment, the diaphragms 120 or 125 are configured as replaceable components. In another embodiment, the head 115 and diaphragm 125 may be integral with each other (e.g., one piece, formed together).

With reference to diaphragm 120, the diaphragm includes a recessed cavity/receptacle that is configured to receive and contain a conductive medium 140 used during an ultrasound scan. In one embodiment, the conductive medium 140 is a portion of gel (e.g., gel shot, gel pad) that is preconfigured to fit into the cavity and the top surface 142 is exposed, as seen in FIG. 2B where gel 140 is inserted into the diaphragm 120. Likewise, diaphragm 125 includes a cavity to receive a conductive medium 145 when used during a scan.

### Preconfiqured Gel Cover/Cap Embodiment

In another embodiment, with reference to FIGS. 3A and 3B, an adaptor 300 for an ultrasound device is disclosed. The adaptor 300 includes an ultrasound conductive medium configured to operate with a flat or generally flat surface of an ultrasound head (e.g., a head without a gel cavity in the head/diaphragm). FIG. 3A shows one embodiment of a perspective view of the adaptor 300 and FIG. 3B shows a cross-section view of FIG. 3A. In one embodiment, the adaptor 300 is piece of preconfigured ultrasound gel that is formed and shaped as a gel cover 300. The gel cover 300 is configured to be attachable like a cap over the head of an ultrasound device (e.g., a cap, sleeve, or other covering type shape).

As previously stated, the gel cover 300 is created from a liquid or gel-like composition of an ultrasound conductive medium and formed into a preconfigured shape and solidified. In various embodiments, the preconfigured gel can be solidified to different degrees or ranges as desired such as being a soft and flexible object, being a rigid object, or any state in between (e.g., semi-rigid and/or flexible structure). In one embodiment shown in FIG. 3A, the gel cover 300 is formed as a generally circular shaped cap that is configured to attach to a generally circular shaped ultrasound head/probe. Of course, other shapes can be formed based on the shape and configuration of an ultrasound head to be used with the preconfigured gel. For example, each type of ultrasound head may have a customized version of the gel cover 300 made for the device.

With reference to FIGS. 3A and 3B, in one example, the gel cover 300 is configured with an internal cavity 305 to receive the ultrasound head/diaphragm. The gel cover 300 includes one or more sidewalls 310 that define the internal cavity 305 via an opening (e.g., a missing wall, no bottom surface). The sidewalls 310 are configured to surround and attach to the head/diaphragm of an ultrasound device. In one embodiment, side wall 310 is a continuous circular wall that extends out from a closed top surface 315 of the cover 300. The sidewalls 310 may be generally straight and/or curved as they extend out from the top surface 315.

The top surface 315 functions as the interface surface and coupling medium between the ultrasound transducer and an ultrasound target (e.g., a patient) once the ultrasound device is inserted into the cavity 305. The thickness of the closed top surface 315 is predetermined and is a measured amount of gel based on the size and shape of the gel cover 300. The predetermined thickness of the top surface 315 and the solid form of the gel cover 300 provides a consistent and controlled amount of gel (e.g., coupling agent/medium) between the ultrasound transducer and the patient. Thus dispensing inconsistent and unknown amounts of liquid gel during an ultrasound procedure is eliminated.

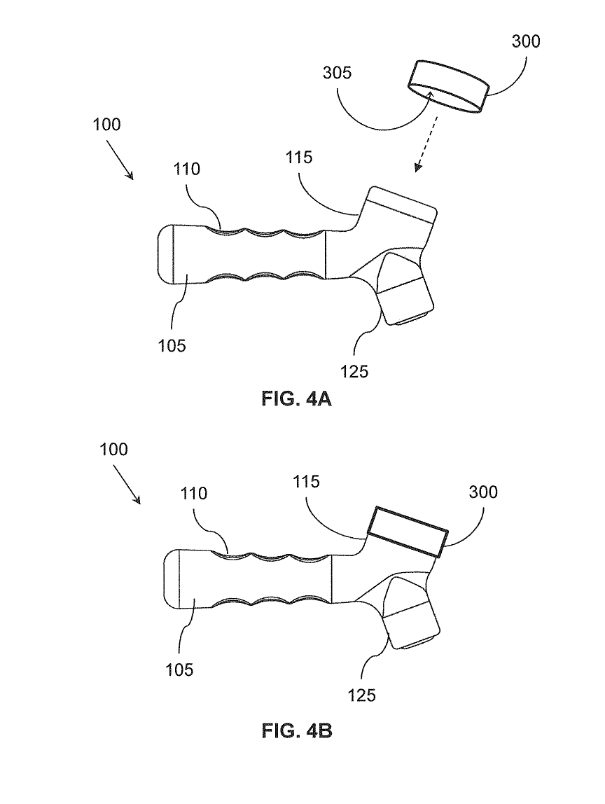

In general, the gel cover 300 is a reverse configuration as shown in FIG. 2A. Instead of the gel 140 inserting into the cavity of the diaphragm 120, here the diaphragm/head is inserted into the gel cover 300. For example, this is shown in FIGS. 4A and 4B where the gel cover 300 is being attached to head 115 in which is at least one ultrasound transducer (FIG. 4A). FIG. 4B shows the gel cover 300 attached to the head 115 (e.g., top of head 115 is inserted into cavity of gel cover 300). The gel cover 300 is preconfigured into a solid form having a shape defined to cover and enclose the head of an ultrasound device. The gel cover 300 is removable and replaceable with other gel covers.

In one embodiment, the gel cover 300 may be a configured similar to a shower-cap design (e.g., cap-like structure) with flexible side walls that can move and stretch to surround, cover, and/or enclose the diaphragm/head of a device. In one embodiment, the gel cover 300 is held in place on the head by at least surface tension, friction from the sidewall(s) 310, and/or the squeezing force from the flexible sidewall(s) 310 against the head. When a scan is complete, the gel cover 300 is simply pulled off and another gel cover can be attached for a subsequent scan. Of course, the gel cover 300 may be used for multiple scans since the cover 300 is attached to the ultrasound device and moves with the device. However, multiple uses of the gel cover 300 may cause issues of sterility that may not be desired. In one embodiment, gel covers may be individually packaged to ensure that each cover 300 is sterile for each use, or a group of gel covers 300 may be packaged together.

In another embodiment with reference to FIG. 3C, the internal surface of the sidewalls 310 may include one or more lips, rings, ribs, edges or other internal protrusions 320. The internal protrusion 320 is configured to lock and/or assist in securing the gel cover 300 onto the head of the ultrasound device. For example, the internal protrusions are oriented and configured to mate with or abut against corresponding receiving notches or edges in the head of the ultrasound device for which the gel cover 300 is made. One or more internal protrusions 320 can be configured within the gel cover 300. A protrusion may be continuously around the internal surface like a ring, vertical and/or horizontal ribs (where horizontal is parallel to the top surface 315 as shown by protrusion 320), or other combination of protrusions. Other sizes and shapes of the protrusion(s) 320 may also be formed to function as a locking element that assists to secure the gel cover 300 to an ultrasound head.

In another embodiment, the sidewalls 310 are configured to fit around the head/diaphragm (e.g., see FIGS. 3B and 4B, generally a U-shaped cross-section) so that when the ultrasound head is inserted into the cavity 305, the gel cover 300 is attached to the device (see FIG. 4B). Thus when the ultrasound device 100 is moved by an operator, the gel cover 300 is moved/pushed along with the device 100 to slide along a patient to provide a consistent and controlled amount of coupling agent-conductive medium/gel between the ultrasound transducer and the patient, which is provided by the interface top surface 315. Thus as stated previously, squirting and spreading random amounts of liquid gel on a patient is eliminated.

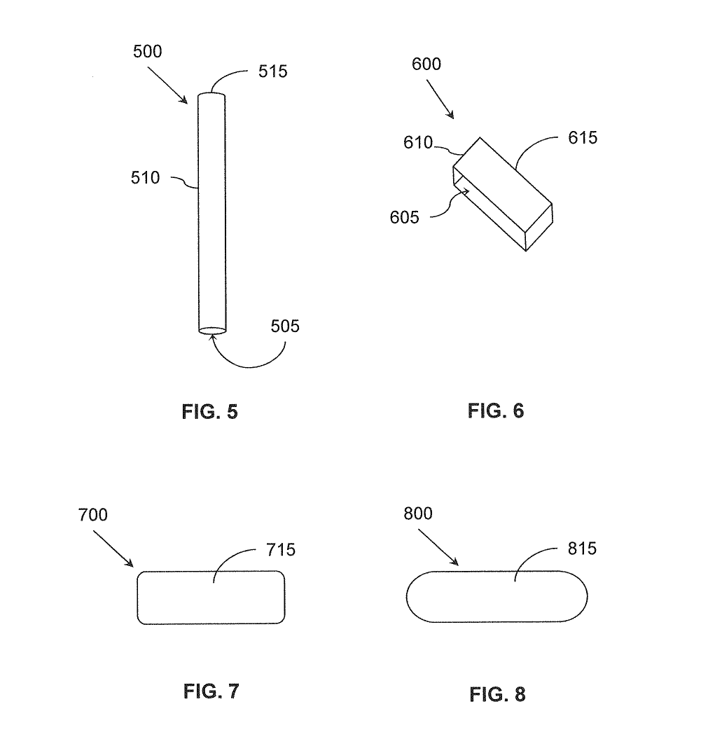

In other embodiments, the gel cover can be formed in other shapes and configurations. For example, with reference to FIG. 5, a gel cover 500 is illustrated as a tube-like structure where sidewalls 510 are elongated and much longer than the sidewalls 310 shown in FIG. 3A. Gel cover 500 is configured to fit a long and narrow ultrasound probe (e.g., stick-like probe). The sidewall(s) 510 defines an internal cavity 505 where the internal cavity 505 is configured to receive a long and narrow ultrasound probe. The gel cover 510 also includes a closed top surface 515 that functions as an interface surface for the ultrasound.

Of course, other shapes can be implemented based on the shape of the ultrasound device. For example, FIG. 6 shows one embodiment of a rectangular shaped gel cap 600 that includes a cavity 605 defined by four sidewalls 610 and a closed top surface 615. FIG. 7 shows another embodiment of a gel cover 700 from a top surface view where the top closed surface 715 is generally rectangular but with rounded corners. FIG. 8 shows another embodiment of a gel cover 800 from a top surface view where the top closed surface 815 is generally oval in shape. In general, the gel cover can be formed to correspond to a particular shape of an ultrasound head so that each type of head can operate with a custom shaped gel cover.

Definitions

The following includes definitions of selected terms employed herein. The definitions include various examples and/or forms of components that fall within the scope of a term and that may be used for implementation. The examples are not intended to be limiting. Both singular and plural forms of terms may be within the definitions.

The term “conductive medium” is used to refer to a substance that is used during an ultrasound procedure that assists in coupling the ultrasound device/probe head or applicator tip to a subject/target (e.g., the skin of a patient or other surface) and conducts ultrasound energy. Typically, the conductive medium is ultrasound gel but other substances can be used such as shampoo, hairstyling gel, hand lotion, hand sanitizer, liquid dishwashing detergent, olive oil (or other oil based substances), or other substance with a composition that is appropriate to function with an ultrasound device. Many substances can form gels when a suitable thickener or gelling agent is added to their formula to change the viscosity. These substances are preconfigured into a solidified state as an individual piece of conductive medium as explained previously (e.g., solidified state may be any state in which the gel holds its shape and does not flow). References to the term “gel” is intended to refer to any of these conductive media that is appropriate for an ultrasound procedure.

References to “one embodiment”, “an embodiment”, “one example”, “an example”, and so on, indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in one embodiment” does not necessarily refer to the same embodiment, though it may.

While example systems, methods, and so on have been illustrated by describing examples, and while the examples have been described in considerable detail, it is not the intention of the applicant to restrict or in any way limit the scope of the appended claims to such detail. It is, of course, not possible to describe every conceivable combination of components or methodologies for purposes of describing the systems, methods, and so on described herein. Therefore, the disclosure is not limited to the specific details, the representative apparatus, and illustrative examples shown and described. Thus, this disclosure is intended to embrace alterations, modifications, and variations that fall within the scope of the appended claims.

To the extent that the term “includes” or “including” is employed in the detailed description or the claims, it is intended to be inclusive in a manner similar to the term “comprising” as that term is interpreted when employed as a transitional word in a claim.

To the extent that the term “or” is used in the detailed description or claims (e.g., A or B) it is intended to mean “A or B or both”. When the applicants intend to indicate “only A or B but not both” then the phrase “only A or B but not both” will be used. Thus, use of the term “or” herein is the inclusive, and not the exclusive use. See, Bryan A. Garner, A Dictionary of Modern Legal Usage 624 (2d. Ed. 1995).

What is claimed is:

1. An ultrasound conductive medium formed into a preconfigured shape that is a cap-like structure configured to attach over a head of an ultrasound device.

2. The ultrasound conductive medium of claim 1, wherein the ultrasound conductive medium is in a solidified state that maintains the preconfigured shape.

3. The ultrasound conductive medium of claim 1, wherein the ultrasound conductive medium is formed from a gel.

4. The ultrasound conductive medium of claim 1, wherein the cap-like structure includes flexible side walls that define an internal cavity.

5. The ultrasound conductive medium of claim 4, wherein the flexible side walls include one or more protrusions formed within the internal cavity, wherein the one or more protrusions are configured to assist in securing the ultrasound conductive medium to the head of the ultrasound device once attached thereto.

6. The ultrasound conductive medium of claim 5, wherein the one or more internal protrusions are oriented and configured to mate with or abut against corresponding notches or edges in the head of the ultrasound device.

7. The ultrasound conductive medium of claim 1, wherein the cap-like structure includes sidewalls and an interface surface, wherein the interface surface has a predetermined thickness of the ultrasound conductive medium to provide a consistent amount of ultrasound conductive medium between the ultrasound device and a patient.

8. An ultrasound conductive medium molded into a solid form having a shape defined to cover and enclose a head of an ultrasound device.

9. The ultrasound conductive medium of claim 8, wherein the ultrasound conductive medium includes one or more sidewalls that define an internal cavity for receiving the head of the ultrasound device.

10. The ultrasound conductive medium of claim 9, wherein the one or more sidewalls include one or more protrusions, wherein the one or more protrusions are configured to assist in securing the ultrasound conductive medium to the head of the ultrasound device once attached thereto.

11. The ultrasound conductive medium of claim 8, wherein the solid form includes a predetermined thickness of ultrasound conductive medium to provide a consistent and controlled amount of the ultrasound conductive medium between a transducer of the ultrasound device and a patient.

12. The ultrasound conductive medium of claim 8, wherein the shape is a cap-like structure that includes flexible sidewalls for attaching to the head of the ultrasound device.

13. The ultrasound conductive medium of claim 8, wherein the shape is a tube shape that includes elongated sidewalls that define an internal cavity, wherein the internal cavity is configured to receive a long and narrow ultrasound probe.

14. The ultrasound conductive medium of claim 8, wherein the cap-like structure includes sidewalls and an interface surface, wherein the interface surface has a predetermined thickness of the ultrasound conductive medium to provide a consistent amount of ultrasound conductive medium between the ultrasound device and a patient.

15. The ultrasound conductive medium of claim 8, wherein the ultrasound conductive medium comprises an ultrasound gel.

16. The ultrasound conductive medium of claim 8, wherein the flexible side walls include one or more protrusions formed within the internal cavity, wherein the one or more protrusions are configured to assist in securing the ultrasound conductive medium to the head of the ultrasound device once attached thereto.

17. An ultrasound device, comprising:
at least a handle connected to a head, wherein the head contains an ultrasound transducer; and
an ultrasound conductive medium having a solidified shape, wherein the solidified shape is attach to and covers the head of the ultrasound device;
wherein the ultrasound conductive medium is configured to provide an interface surface and coupling medium between the ultrasound transducer and an ultrasound target.

18. The ultrasound device of claim 17, wherein the solidified shape is a cap-like structure comprising one or more flexible sidewalls that define an internal cavity for receiving the head of the ultrasound device, wherein the ultrasound conductive medium is attached to move with the head when the ultrasound device is moved.

19. The ultrasound device of claim 18, wherein the one or more sidewalls includes one or more protrusions.

20. The adaptor of claim 17, wherein the solidified shape comprises solidified gel and wherein the ultrasound conductive medium is removable from the head.
